(19) United States
(12) Patent Application Publication
(10) Pub. No.: US 2022/0174983 A1
(43) Pub. Date: Jun. 9, 2022

(54) PROBIOTICS FOR REDUCING METHANE PRODUCTION

(71) Applicant: SUPERBREWED FOOD, INC., New Castle, DE (US)

(72) Inventors: Bryan P. TRACY, Wilmington, DE (US); Alon KARPOL, Tel-Mond (IL); Aharon M. EYAL, Jerusalem (IL)

(21) Appl. No.: 17/436,085

(22) PCT Filed: Mar. 4, 2020
(86) PCT No.: PCT/IB2020/051819

§ 371 Date: Sep. 3, 2021

Related U.S. Application Data
(60) Provisional application No. 62/814,239, filed on Mar. 5, 2019.
(60) Provisional application No. 62/964,135, filed on Jan. 22, 2020.

(51) Int. Cl. A23K 10/18; A23K 20/105; A23K 50/10; A61K 45/06; A61K 35/741

(52) U.S. Cl. CPC A23K 10/18; A23K 20/105; A61K 35/741; A61K 45/06; A23K 50/10

(57) ABSTRACT

Provided is a probiotic composition comprising at least one organism and a carrier, which organism is characterized by having the ability to metabolize carbohydrates and/or their products and by production of less than 0.5 mole hydrogen per mole of metabolized carbohydrate equivalent, wherein said composition is characterized by reducing methane production when administered to a ruminant. Also provided are methods of use of the composition in reducing methane production by a ruminant.

### SUMMARY OF THE INVENTION

According to an aspect of some embodiments of the present invention, provided is a probiotic composition comprising at least one organism and a carrier, which organism is characterized by having the ability to metabolize carbohydrates and/or their products and by production of less than 0.5 mole hydrogen per mole of metabolized carbohydrate equivalent, wherein the composition is characterized by reducing methane production when administered to a ruminant.

According to a further aspect of some embodiments of the invention, there is provided a method for reducing production of methane emanating from digestive activities of a ruminant, the method comprising administering to said ruminant at least one organism and a carrier, which organism is characterized by having the ability to metabolize carbohydrates and/or their products and by production of less than 0.5 mole hydrogen per mole of metabolized carbohydrate equivalent.

### DETAILED DESCRIPTION OF THE INVENTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “probiotic” refers to a live microorganism which provides health benefits to an animal when consumed, generally by restoring the balance of gut flora.

As used herein, the term “methanogenesis inhibitor” refers to a compound having the ability to block methane production by methanogenic bacteria. In some embodiments, methane production is blocked by inhibition of the last enzyme in the methane production pathway. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, at least 95% or even 100% of methane production is blocked as compared to methane production in the absence of the methanogenesis inhibitor.

As used herein, the term “vegetative culture” refers to a culture comprising cells which are able to grow, divide, metabolize and move.

As used herein, the term “syntrophic behavior” refers to a nutritional interdependence between two or more microorganisms. Such interdependence may be mutually beneficial but is not necessarily required for growth. According to an embodiment, the syntrophic behavior involves a first microorganism consuming a metabolite produced by a second microorganism, wherein the metabolite is harmful to the environment or to the second microorganism, resulting in the removal or reduction of the level of the harmful metabolite.

According to an embodiment, the syntrophic behavior occurs between one or more microorganisms within the mixture of microorganisms in the composition. Alternatively or additionally, the syntrophic behavior occurs between one or more microorganisms in the composition and one or more native microorganisms in the animal to which the composition is administered.

As used herein, the term “obligate anaerobic growth” refers to the ability of an organism to grow, divide, move and metabolize in the absence of oxygen, or in the presence of less than 10% (v/v) of oxygen.

As used herein, the term “tolerance to bile salts” refers to the ability to divide, grow and metabolize in the presence of bile salts wherein the levels of division, growth and metabolism in the presence of at least 0.1 wt % of bile salts are at least 50% that of the organism in the absence of bile salt and/or to survive with at least 50% cell recovery after exposure to at least 0.1 wt % of bile salts for 2 hours in a solution having the same composition as that of gastric fluids. As used herein, the term “being capable of producing a cellulose-hydrolyzing enzyme” refers to an organism in which at least 1 wt % of the total cell proteins are cellulases and/or in which cellulase activity is at least 1 filter paper unit (FPU) per 10⁹ cell forming units (CFU).

As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

As used herein, when a numerical value is preceded by the term “about”, the term “about” is intended to indicate +/−10% of that value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an embodiment, provided is a probiotic composition comprising at least one organism and a carrier, which organism is characterized by having the ability to catabolize carbohydrates and/or their products and by production of less than 0.5 mole hydrogen per mole of metabolized carbohydrate equivalent, wherein the composition is characterized by reducing methane production when administered to a ruminant.

According to an embodiment, the organism has the ability to metabolize carbohydrates at a rate of at least 0.01 g/hr/g cell mass, at least 0.02, at least 0.03, at least 0.04, at least 0.05, at least 0.1, at least 0.15, at least 0.2 or at least 0.25 g/hr/g cell mass.

According to an embodiment, provided is a probiotic composition characterized by reducing methane production when administered to a ruminant.

According to an embodiment, said organism is characterized by having the ability to metabolize carbohydrates and/or their products and by production of less than 0.5 mole hydrogen per mole of metabolized carbohydrate equivalent. According to an embodiment, said organism is capable of metabolizing carbohydrates and products thereof comprising monosaccharides, disaccharides, oligosaccharides and polysaccharides, such as starch, cellulose and hemicellulose, as well as products of carbohydrates fermentation, e.g. glycerol and organic acids. According to an embodiment, said organism is further characterized by producing no hydrogen on metabolizing said carbohydrates and/or products thereof. According to an alternative embodiment, said organism is characterized by producing of less than 0.5 mole hydrogen per mole of metabolized carbohydrate equivalent, less than 0.4 mole, less than 0.3 mole, less than 0.2 mole, or less than 0.1 mole.

According to an embodiment, the at least one organism comprises a mixture of organisms.

According to an embodiment, the at least one organism is further characterized by having at least one property selected from the group consisting of being capable of producing a cellulose-hydrolyzing enzyme, having a maximal growth rate constant of at least 2.0 hour⁻¹, being capable of non-oxidative glycolysis, being capable of consuming lactate at a rate greater than 0.01 g/hr/g cell mass and forming at least one of acetate and/or butyrate at a rate greater than 0.01 g/hr/g cell mass, being capable of consuming lactate at a rate greater than 0.01 g/hr/g cell mass and forming propionate at a rate greater than 0.01 g/hr/g cell mass and being capable of producing at least 0.1 g/L of an organic acid or a salt thereof. According to an embodiment, a cellulose-hydrolyzing enzyme is selected from the group consisting of endo-cellulase, exo-cellulase, beta-glucosidase, lytic polysaccharide monooxygenase (LPMO) and an enzyme from the glycosyl hydrolyze (GH) family.

According to an embodiment, the organism is further characterized by producing at least 0.1 g/L of an organic acid or salt thereof, wherein said organic acid is selected from the group consisting of propionic acid, butyric acid, lactic acid, formic acid, acetic acid, oxalic acid and combinations thereof.

According to an embodiment, the organism is further characterized by having at least one property selected from the group consisting of butanoate metabolism, obligate anaerobic growth, gas fixation via the reductive acetyl-coenzyme A pathway, tolerance to bile salts at concentration greater than 0.05%, tolerance to pH of less than 3.5, and self-aggregation.

According to an embodiment, butanoate metabolism comprises phosphorylation of butyryl CoA to form butyryl phosphate, followed by conversion of butyryl phosphate to butyrate by the action of butyrate kinase. According to an alternative embodiment, butanoate metabolism comprises transfer of the CoA moiety of butyryl CoA to acetate by the action of butyryl CoA-acetate CoA transferase, resulting in the formation of butyrate and acetyl CoA.

According to an embodiment, said organism is characterized by having at least two of said properties, at least three, at least four at least five or at least six. According to an embodiment, said organism comprises a mixture of organisms, which mixture comprises organisms characterized by one or several of said properties and organisms characterized by an additional property or additional properties.

According to an embodiment, the composition further comprises a methanogenesis inhibitor.

According to an embodiment, said methanogenesis inhibitor comprises an inhibitor of the enzyme methyl coenzyme M reductase (MCR).

According to an embodiment, said inhibitor of the enzyme methyl coenzyme M reductase comprises 3-Nitrooxypropanol (3-NOP).

According to an embodiment, the composition further comprises a nitrate reducing organism.

According to an embodiment, the composition comprises a live vegetative culture of the organism.

According to an embodiment, the composition comprises a sporulated culture of the organism.

According to an embodiment, the mixture of organisms is a syntrophic mixture showing syntrophic behavior.

According to an embodiment, the syntrophic behavior is beneficial to an animal consuming the composition.

According to an embodiment, the mixture of organisms comprises at least one CO₂-utilizing organism.

According to an embodiment, the CO₂-utilizing organism is an acetogen.

According to an embodiment, the organism is selected from the group consisting of Acetitomaculum ruminis; Acetobacterium carbinolicum; Acetobacterium psammolithicum; Acetobacterium woodii; Bacillus megaterium; Bacillus subtilis; Bacteroides fragilis; Blautia producta; Clostridium aceticum; Clostridium acidiurici; Clostridium cylindrosporum; Clostridium formicaceticum; Clostridium magnum; Clostridium pasteurianum; Clostridium perfringens; Clostridium sardiniense; Desulfovibrio piger; Enterococcus faecalis; Escherichia coli; Gottschalkia acidurici; Methylobacterium extorquens; Micrococcus aerogenes; Micrococcus luteus; Moorella thermoacetica; Moraxella catarrhalis; Mycobacterium smegmatis; Neurospora crassa; Oxobacter pfennigii; Peptoniphilus asaccharolyticus; Peptostreptococcus anaerobius; Proteus mirabilis; Proteus vulgaris; Reticulitermes santonensis; Rhizobium leguminosarum; Saccharomyces cerevisiae; Sinorhizobium medicae; Sinorhizobium meliloti; Sphingomonas paucimobilis; Sporomusa ovata; Sporomusa termitida; Staphylococcus epidermidis; Thermacetogenium phaeum; Thermoplasma acidophilum; Treponema denticola; Treponema primitia; Veillonella parvula and combinations thereof.

According to an embodiment, said at least one organism is selected from the group consisting of Acetobacterium woodii; Bacillus megaterium; Bacillus subtilis; Bacteroides fragilis; Blautia producta; Clostridium aceticum; Clostridium acidiurici; Clostridium cylindrosporum; Clostridium formicaceticum; Clostridium magnum; Clostridium pasteurianum; Enterococcus faecalis; Moorella thermoacetica; Moraxella catarrhalis; Peptostreptococcus anaerobius; Treponema primitia; Veillonella parvula and combinations thereof.

According to an embodiment, said at least one organism comprises Acetobacterium woodii. According to an embodiment, said at least one organism comprises Bacillus megaterium. According to an embodiment, said at least one organism comprises Bacillus subtilis. According to an embodiment, said at least one organism comprises Bacteroides fragilis. According to an embodiment, said at least one organism comprises Blautia product. According to an embodiment, said at least one organism comprises Clostridium aceticum. According to an embodiment, said at least one organism comprises Clostridium acidiurici According to an embodiment, said at least one organism comprises Clostridium cylindrosporum. According to an embodiment, said at least one organism comprises Clostridium formicaceticum. According to an embodiment, said at least one organism comprises Clostridium magnum. According to an embodiment, said at least one organism comprises Clostridium pasteurianum. According to an embodiment, said at least one organism comprises Enterococcus faecalis. According to an embodiment, said at least one organism comprises Moorella thermoacetica. According to an embodiment, said at least one organism comprises Moraxella catarrhalis. According to an embodiment, said at least one organism comprises Peptostreptococcus anaerobius. According to an embodiment, said at least one organism comprises Treponema primitia. According to an embodiment, said at least one organism comprises Veillonella parvula.

According to an embodiment, the carrier is selected from the group consisting of water, saline, aqueous dextrose, lactose, a buffered solution, starch, cellulose, glucose, lactose, sucrose, gelatin, malt, rice, flour and combinations thereof.

According to an embodiment, there is provided a feed comprising the composition as disclosed herein.

According to an embodiment, the feed further comprises enzymes selected from the group consisting of cellulolytic enzymes: endoglucanase, exoglucanase, beta glucosidase, Lytic polysaccharide monooxygenases, Xylosidase and combinations thereof.

According to an aspect of some embodiments of the present invention, there is provided a method for reducing production of methane emanating from digestive activities of a ruminant, the method comprising administering to said ruminant at least one organism and a carrier, which organism is characterized by having the ability to metabolize carbohydrates and/or their products and by production of less than 0.5 mole hydrogen per mole of metabolized carbohydrate equivalent.

According to an embodiment, said ruminant is fed cellulose-comprising feed and wherein hydrogen production in the rumen of said ruminant prior to administering said organism is less than 24.4 grams per one kilogram of fed cellulose.

According to an embodiment, the ruminant is fed cellulose-comprising feed and wherein hydrogen production in the rumen of the ruminant is less than 24.4 grams per one kilogram of fed cellulose.

According to an embodiment, the ruminant is fed a given amount of cellulose-comprising feed and wherein methane production is reduced by at least 2% compared with methane production on feeding a same amount of the feed in the absence of administering of said organism such as at least 2%, at least 5%, at least 10%, at least 15%, at least 20%, at least 25%, at least 30%, at least 35%, at least 40%, at least 45% or even at least 50%.

According to an embodiment, the ruminant is fed a given amount of cellulose-comprising feed for energy and wherein energy production is increased by at least 2% compared with energy production on feeding a same amount of said feed in the absence of administering of said organism, such as at least 2%, at least 5%, at least 10%, at least 15%, at least 20%, at least 25%, at least 30%, at least 35%, at least 40%, at least 45% or even at least 50%.

According to an embodiment, said administering is carried out under conditions which allow colonizing the rumen of said ruminant with said organism

According to an embodiment, said colonizing provides from about 10² to about to about 10¹⁰ Colony-Forming Units per gram wet feces of said ruminant on the twentieth day after administering

According to an embodiment, the colonizing results in an organic acid concentration of at least 0.1 millimolar in said rumen.

According to an embodiment, said colonizing results in forming organic acid at a rate of at least 0.01 millimole per hour.

According to an embodiment, said ruminant is selected from the group consisting of Cow; Bull; Ox; Bison; Buffalo; Eland; Four-horned antelope; Water buffalo; Wild yak; Yak; Sheep; Bighorn sheep; Domestic sheep; Snow sheep; Trinhorn sheep; Urial; Goat; Alpine ibex; Bharal; Barbary sheep; Chamois; Chinese goral; Chinese serow; Dwarf blue sheep; Markhor; Mountain goat; Nubian ibex; Siberian ibex; Spanish ibex; Walia ibex; Deer; Elk; Eld's deer; Fallow deer; Hog deer; Moose; Red deer; Reindeer; and Caribou.

According to an embodiment, the method further comprises administering a methanogenesis inhibitor.

According to an embodiment, the organism and the methanogenesis inhibitor are provided in a single dosage form.

According to an embodiment, the organism and the methanogenesis inhibitor are provided in separate dosage forms, for simultaneous or sequential administration. According to one such embodiment, the organism is administered one, two, three, four or five times in total, preferably when the ruminant is young, while the inhibitor is administered repeatedly during the lifetime of the ruminant. In some embodiments, the organism is administered daily. In some embodiments, the organism is administered weekly. In some embodiments, the organism is administered bi-weekly. In some embodiments, the organism is administered monthly.

According to an embodiment, said organism is administered by adding to the feed of the animal.

According to an embodiment, said methanogenesis inhibitor is administered by adding to the feed of the animal.

According to an embodiment, said organism and said methanogenesis inhibitor are administered by adding to the same feed of the animal.

According to an embodiment, said organism and said methanogenesis inhibitor are administered by adding to different feeds of the animal.

According to an embodiment, said methanogenesis inhibitor is administered by adding to the drinking water of the animal.

According to a preferred embodiment, the organism is administered prior to administration of the inhibitor.

According to an embodiment, the inhibitor is administered according to methods known in the art, such as described in U.S. Pat. Nos. 10,154,981; 9,902,685; and 9,266,814 which are incorporated by reference as if fully set out herein.

According to an embodiment, administration of both the organism and the inhibitor provides a synergistic effect as compared to the effect obtained by use of either the organism or the inhibitor alone. According to an embodiment, the synergistic effect comprises an effect that is greater than the sum of the individual effects of the organism alone or the inhibitor alone.

According to an embodiment, the synergistic effect provides an increase of at least two-fold, at least three-fold, at least four-fold, at least five-fold, at least six-fold, at least seven-fold, at least eight-fold, at least nine-fold, or even at least ten-fold as compared to the effect obtained with the organism alone.

According to an embodiment, the synergistic effect enables a decreased concentration of the organism and/or the inhibitor in order to obtain a same effect as obtained in the absence of either of the organism alone or the inhibitor alone.

According to an embodiment, the synergistic effect enables a reduction in concentration of at least 2%, at least 5%, at least 10%, at least 20%, at last 30%, at least 40% or even at least 50% of the organism and/or the inhibitor to be used in order to obtain a same effect as obtained in the absence of either of the organism alone or the inhibitor alone.

Without wishing to be limited to any one theory, the present inventors hypothesize that administration of the organism and the inhibitor together increases rumen fermentation compared with the fermentation occurring upon administering the inhibitor alone. According to an embodiment, butyric acid is produced by such rumen fermentation. According to an embodiment, butyric acid produced in such rumen fermentation provides an energy source for said ruminant. The present inventors further hypothesize that a limiting factor occurs when either the inhibitor or the organism is administered alone, which is removed by administration of both the inhibitor and the organism. Specifically, 3-NOP has been found to block methane production by inhibiting the last enzyme in the methane production pathway, but the methane is a sink for the electrons, such that without this sink the ruminant accumulates hydrogen, which in turns, affects the equilibrium of other enzymatic pathways and could reduce rumen fermentation. Adding an acetogen that could consume the accumulated hydrogen, thus relieving the high hydrogen concentration in the rumen improves rumen fermentation. On the other the other hand, when only acetogen is administered to compete with methanogen for hydrogen, since hydrogen consumption by methanogens is more thermodynamically favored, acetogens consume very little hydrogen. Inhibiting the methanogen pathway, (by adding 3-NOP) results in diverting the hydrogen for use as a substrate by the acetogens.

According to an embodiment, said methanogenesis inhibitor comprises an inhibitor of the enzyme methyl coenzyme M reductase (MCR).

According to an embodiment, said inhibitor of the enzyme methyl coenzyme M reductase comprises 3-Nitrooxypropanol (3-NOP).

According to an embodiment, production of methane emanating from digestive activities of a ruminant is reduced by at least 2%, such as at least 5%, at least 10%, at least 15%, at least 20%, at least 25%, at least 30%, at least 35%, at least 40%, at least 45%, or even at least 50%, calculated in liters per kilogram of dry matter intake when measured in a metabolic chamber, compared with administering same dose of organism, but not said inhibitor.

According to an embodiment, production of methane emanating from digestive activities of a ruminant upon administering of both of said organism and said inhibitor is reduced by at least 2%, such as at least 5%, at least 10%, at least 15%, at least 20%, at least 25%, at least 30%, at least 35%, at least 40%, at least 45%, or even at least 50%, calculated in liters per kilogram of dry matter intake when measured in a metabolic chamber, compared with a reduction in production of methane emanating from digestive activities of said ruminant obtained by administering a same dose of said organism in the absence of said inhibitor.

According to an embodiment, production of methane emanating from digestive activities of a ruminant upon administering of both of said organism and said inhibitor is reduced by at least 2%, such as at least 5%, at least 10%, at least 15%, at least 20%, at least 25%, at least 30%, at least 35%, at least 40%, at least 45%, or even at least 50%, calculated in liters per kilogram of dry matter intake when measured in a metabolic chamber, compared with a reduction in production of methane emanating from digestive activities of said ruminant obtained by administering a same dose of said inhibitor in the absence of said organism.

According to an embodiment, administering of both of said organism and said inhibitor provide a synergistic effect in reducing said production of methane emanating from digestive activities of a ruminant, as compared with a reduction in production of methane emanating from digestive activities of said ruminant obtained by administering a same dose of said organism in the absence of said methanogenesis inhibitor or a same dose of said methanogenesis inhibitor in the absence of said organism.

According to an embodiment, feed efficiency upon administering of both of said organism and said inhibitor is increased by at least 2%, such as at least 5%, at least 10%, at least 15%, at least 20%, at least 25%, at least 30%, at least 35%, at least 40%, at least 45%, or even at least 50%, compared with an increase in feed efficiency obtained by administering a same dose of said organism in the absence of said inhibitor.

According to an embodiment, feed efficiency upon administering of both of said organism and said inhibitor is increased by at least 2%, such as at least 5%, at least 10%, at least 15%, at least 20%, at least 25%, at least 30%, at least 35%, at least 40%, at least 45%, or even at least 50%, compared with an increase in feed efficiency obtained by administering a same dose of said inhibitor in the absence of said organism.

According to an embodiment, administering of both said organism and said inhibitor provides a synergistic effect in increasing feed efficiency as compared with an increase in feed efficiency obtained by administering a same dose of said organism in the absence of said inhibitor or a same dose of said inhibitor in the absence of said organism.

According to an embodiment, milk production, milk fat or both upon administering of both of said organism and said inhibitor is increased by at least 2%, such as at least 5%, at least 10%, at least 15%, at least 20%, at least 25%, at least 30%, at least 35%, at least 40%, at least 45%, or even at least 50%, compared with an increase in milk production, milk fat or both obtained by administering a same dose of said organism in the absence of said inhibitor.

According to an embodiment, milk production, milk fat or both upon administering of both of said organism and said inhibitor is increased by at least 2%, such as at least 5%, at least 10%, at least 15%, at least 20%, at least 25%, at least 30%, at least 35%, at least 40%, at least 45%, or even at least 50%, compared with an increase in milk production, milk fat or both obtained by administering a same dose of said inhibitor in the absence of said organism.

According to an embodiment, administering of both said organism and said inhibitor provides a synergistic effect in increasing milk production, milk fat or both as compared with an increase in milk production, milk fat or both obtained by administering a same dose of said organism in the absence or said inhibitor, or a same dose of said inhibitor in the absence of said organism.

According to an embodiment, production of butyric acid, production of acetic acid or production of both in the rumen of said ruminant is increased by at least 2%, such as at least 5%, at least 10%, at least 15%, at least 20%, at least 25%, at least 30%, at least 35%, at least 40%, at least 45%, or even at least 50%, compared with an increase in production of butyric acid, production of acetic acid or production of both obtained by administering a same dose of said organism in the absence of said inhibitor.

According to an embodiment, production of butyric acid, production of acetic acid or production of both in the rumen of said ruminant is increased by at least 2%, such as at least 5%, at least 10%, at least 15%, at least 20%, at least 25%, at least 30%, at least 35%, at least 40%, at least 45%, or even at least 50%, compared with an increase in production of butyric acid, production of acetic acid or production of both obtained by administering a same dose of said inhibitor in the absence of said organism.

According to an embodiment, administering of both said organism and said inhibitor provides a synergistic effect in increasing production of butyric acid, production of acetic acid or production of both in the rumen of said ruminant as compared with an increase in production of butyric acid, production of acetic acid or production of both obtained by administering a same dose of said inhibitor in the absence of said organism or a same dose of said organism in the absence of said inhibitor.

According to an embodiment, production of ammonia in the rumen of said ruminant is decreased by at least 2%, such as at least 5%, at least 10%, at least 15%, at least 20%, at least 25%, at least 30%, at least 35%, at least 40%, at least 45%, or even at least 50%, compared with a decrease in production ammonia obtained by administering a same dose of said organism in the absence of said inhibitor.

According to an embodiment, production of ammonia in the rumen of said ruminant is decreased by at least 2%, such as at least 5%, at least 10%, at least 15%, at least 20%, at least 25%, at least 30%, at least 35%, at least 40%, at least 45%, or even at least 50%, compared with a decrease in production of ammonia obtained by administering a same dose of said inhibitor in the absence of said organism.

According to an embodiment, administering of both said organism and said inhibitor provides a synergistic effect in decreasing ammonia production in the rumen of said ruminant as compared with a decrease in production of ammonia obtained by administering a same dose of said inhibitor in the absence of said organism or a same dose of said organism in the absence of said inhibitor.

### Example

Four groups of dairy cows are tested as follows:

Group I: Cells of Eubacterium limosum, a Clostridia class microorganism, are added to the feed at a concentration of 10⁶ colony forming units/Kg of feed for 2 consecutive days. This group further receives 3-Nitrooxypropanol (3-NOP) at the concentration of 5 grams/Kg feed for the full duration of the study.
Group II: The cows receive 3-Nitrooxypropanol (3-NOP) at a concentration of 5 grams/Kg feed for the full duration of the study. No Eubacterium limosum cells are added to the feed.
Group III: Cells of Eubacterium limosum, a Clostridia class microorganism, are added to the feed at a concentration of 10⁶ colony forming units/Kg of feed for 2 consecutive days. No 3-NOP is administered.
Group IV: No Eubacterium limosum or 3-NOP are given (control group).
All other conditions and treatments (provision of vaccination, vitamins, minerals, de-warming, water, antibiotics, etc.) were the same for each of the groups.

In group I, two days after administration of Eubacterium limosum and 3-NOP, methane production decreases by 10% as compared to the control. In parallel, dairy production increases by 2% and the fat content in the milk increases by 4% as compared to the control.

In group II, two days after administration of 3-NOP, methane reduction decreases by 10% as compared to the control, but milk yield and fat content are the same as those of the control.

In group III, two days after administration of Eubacterium limosum, methane production is the same as that of the control, milk yield and fat content in the milk are increased as compared to the control.

1. A probiotic composition comprising at least one organism and a carrier, which organism is characterized by having the ability to metabolize carbohydrates and/or their products and by production of less than 0.5 mole hydrogen per mole of metabolized carbohydrate equivalent, wherein said composition is characterized by reducing methane production when administered to a ruminant,
wherein said at least one organism optionally comprises a mixture of organisms,
wherein said at least one organism is optionally further characterized by having at least one property selected from the group consisting of being capable of producing a cellulose-hydrolyzing enzyme, having a maximal growth rate constant of at least 2.0 hour⁻¹; being capable of non-oxidative glycolysis; being capable of consuming lactate and forming at least one of acetate and butyrate; being capable of consuming lactate and forming propionate; being capable of producing an organic acid or a salt thereof; producing an organic acid or salt thereof, wherein said organic acid is selected from the group consisting of propionic acid, butyric acid, lactic acid, formic acid, acetic acid, oxalic acid and combinations thereof; having at least one property selected from the group consisting of butanoate metabolism, obligate anaerobic growth, gas fixation via the reductive acetyl-coenzyme A pathway, tolerance to bile salts at concentration greater than 0.05%, tolerance to pH of less than 3.5, and self-aggregation.

2-5. (canceled)

6. The composition of claim 1, further comprising a methanogenesis inhibitor.

7. The composition of claim 6, wherein said methanogenesis inhibitor comprises an inhibitor of the enzyme methyl coenzyme M reductase (MCR), wherein said inhibitor of the enzyme methyl coenzyme M reductase optionally comprises 3-Nitrooxypropanol (3-NOP).

8. (canceled)

9. The composition of claim 1, further comprising a nitrate reducing organism.

10. The composition of claim 1, comprising a live culture of said at least one organism, wherein said culture is a vegetative and/or sporulated culture.

11. (canceled)

12. The composition of claim 1, wherein said mixture of organisms is a syntrophic mixture showing syntrophic behavior.

13. (canceled)

14. The composition of claim 1, wherein said at least one organism is a mixture of organisms comprising at least one CO₂-utilizing organism, wherein said CO₂-utilizing organism is optionally an acetogen.

15. (canceled)

16. The composition of claim 1, wherein said at least one organism is selected from the group consisting of Acetitomaculum ruminis; Acetobacterium carbinolicum; Acetobacterium psammolithicum; Acetobacterium woodii; Bacillus megaterium; Bacillus subtilis; Bacteroides fragilis; Blautia producta; Clostridium aceticum; Clostridium acidiurici; Clostridium cylindrosporum; Clostridium formicaceticum; Clostridium magnum; Clostridium pasteurianum; Clostridium perfringens; Clostridium sardiniense; Desulfovibrio piger; Enterococcus faecalis; Escherichia coli; Gottschalkia acidurici; Methylobacterium extorquens; Micrococcus aerogenes; Micrococcus luteus; Moorella thermoacetica; Moraxella catarrhalis; Mycobacterium smegmatis; Neurospora crassa; Oxobacter pfennigii; Peptoniphilus asaccharolyticus; Peptostreptococcus anaerobius; Proteus mirabilis; Proteus vulgaris; Reticulitermes santonensis; Rhizobium leguminosarum; Saccharomyces cerevisiae; Sinorhizobium medicae; Sinorhizobium meliloti; Sphingomonas paucimobilis; Sporomusa ovata; Sporomusa termitida; Staphylococcus epidermidis; Thermacetogenium phaeum; Thermoplasma acidophilum; Treponema denticola; Treponema primitia; Veillonella parvula and combinations thereof.

17. (canceled)

18. A feed comprising the composition of claim 1.

19. The feed of claim 18, further comprising at least one enzyme selected from the group consisting of cellulolytic enzymes: endoglucanase, exoglucanase, beta glucosidase, Lytic polysaccharide monooxygenases, Xylosidase and combinations thereof.

20. A method for reducing production of methane emanating from digestive activities of a ruminant, the method comprising administering to said ruminant at least one organism and a carrier, which organism is characterized by having the ability to metabolize carbohydrates and/or their products and by production of less than 0.5 mole hydrogen per mole of metabolized carbohydrate equivalent.

21. The method of claim 20, wherein said ruminant is fed cellulose-comprising feed and wherein hydrogen production in the rumen of said ruminant prior to administering said organism is less than 24.4 grams per one kilogram of fed cellulose.

22. The method of claim 20, wherein said ruminant is fed a given amount of cellulose-comprising feed and wherein said methane production is reduced by at least 2% compared with methane production on feeding a same amount of said feed in the absence of said administering of said organism.

23. The method of claim 20, wherein said ruminant is fed a given amount of cellulose-comprising feed for energy and wherein energy production is increased by at least 2% compared with energy production on feeding a same amount of said feed in the absence of said administering of said organism.

24. The method of claim 20, wherein said administering comprises administering a concentration of 10⁶ colony forming units/Kg of feed for 2 consecutive days which allows colonizing the rumen of said ruminant with said organism.

25. (canceled)

26. The method of claim 24, wherein said colonizing results in an organic acid concentration of at least 0.1 millimolar in said rumen.

27. The method of claim 24, wherein said colonizing results in forming organic acid at a rate of at least 0.01 millimole per hour.

28. (canceled)

29. The method of claim 20, further comprising administering a methanogenesis inhibitor, wherein administering of said organism and said methanogenesis inhibitor optionally provides a synergistic effect.

30. The method of claim 29, wherein said methanogenesis inhibitor comprises an inhibitor of the enzyme methyl coenzyme M reductase (MCR), wherein said inhibitor of the enzyme methyl coenzyme M reductase optionally comprises 3-Nitrooxypropanol (3-NOP).

31-32. (canceled)

33. The method of claim 29, wherein administering of both said organism and said inhibitor provides at least one synergistic effect selected from the group consisting of increasing feed efficiency, increasing milk production, increasing milk fat, increasing butyric acid production, increasing acetic acid production, decreasing ammonia production or combinations thereof as compared with an increase in feed efficiency obtained by administering a same dose of said organism in the absence of said inhibitor or a same dose of said inhibitor in the absence of said organism.

34-36. (canceled)
